(19) United States
(12) Patent Application Publication
(10) Pub. No.: US 2016/0038032 A1
(43) Pub. Date: Feb. 11, 2016

(54) Pre-Intubation Examination Apparatus and Probe

(71) Applicant: Truphatek International Ltd., Netanya (IL)

(72) Inventor: Gabriel Dan, Tel Aviv (IL)

(21) Appl. No.: 14/779,309

(22) PCT Filed: Mar. 21, 2014
(86) PCT No.: PCT/IL14/50312

§ 371 Date: Sep. 22, 2015

(30) Foreign Application Priority Data
Mar. 24, 2013 (IL) 225445

(51) Int. Cl. A61B 5/00; A61B 1/267

(52) U.S. Cl. CPC A61B 5/0088; A61B 5/0077; A61B 5/742; A61B 5/7475; A61B 1/267; A61B 2019/507

(57) ABSTRACT

Pre-intubation examination apparatus and method for generating digital pre-intubation examination files for assisting successful intubations. The digital pre-intubation examination files include personal patient details, facial photographic data, one or more standard exterior patient anatomical measurements determined from the facial photographic data, and a clinician's subjective rating of a patient on one or more of the standard intubation classifications. The pre-intubation examination apparatus can be implemented either as a standalone pre-intubation examination probe or a pre-intubation examination accessory for use with a commercially available handheld digital device, for example, a computer tablet.

### FIELD OF THE INVENTION

The present invention relates to medical examinations in general and pre-intubation medical examinations in particular.

### BACKGROUND OF THE INVENTION

When an intubation procedure is to be performed on a patient, for example, with the aid of a laryngoscope, it is often desirable to pre-operatively examine the patient to subjectively grade him from being “easy-to-intubate” to “difficult-to-intubate”. Pre-intubation medical examinations are often impractical in emergency situations but are common prior to scheduled operations. A clinician performs a visual examination of a patient for subjectively rating him on one or more of three standard intubation classifications. A clinician also preferably takes exterior patient anatomical measurements which have been found to correlate with rating patients from being “easy-to-intubate” to “difficult-to-intubate”.

The three standard intubation classifications requiring a clinician's subjective rating of a patient are the Mallampati classification for classifying the anatomy of a patient's oral cavity, the upper lip bite classification for classifying the distance to which a patient can bite his upper lip with his lower incisors, and the Cormack-Lehane classification for classifying a patient's laryngeal aperture.

The four standard exterior patient anatomical measurements are a patient's ThyroMental Distance (TMD) defined as the distance from the tip of his thyroid cartilage to the tip of his chin, a patient's SternoMental Distance (SMD) defined as the distance from the upper border of his manubrium to the tip of his mandible, a patient's Atlanto-Occipital Joint Extension (AOJE) defined as the angle a patient can raise his head from a forward facing position, and a patient's Inter Incisor Gap (IIG) defined as the distance between his two lower incisors.

Once the visual examination has been completed and the standard exterior patient anatomical measurements have been taken, the clinician may summarize his findings on a standard form provided by the healthcare facility in which the operation is to be performed. The clinician examining a patient and completing the standard form may not perform the intubation procedure itself in which case the standard form can serve as a quick reference for the clinician performing the intubation procedure.

U.S. Pat. No. 8,460,215 to Connor et al. entitled Systems and Methods for Predicting Potentially Difficult Intubations of a Subject employ a facial structure analysis system for acquiring facial structure data of a patient that is relative in nature rather than objective distances of exterior patient anatomical measurements. The systems and methods also extract, using facial structure analysis software of the facial structure analysis system, at least one facial structure variable value from the facial structure data and obtain a predicted potential difficult intubation value using an airway classification model and the at least one facial structure variable value. The systems and methods generate a report based on the predicted potential difficult intubation value.

### SUMMARY OF THE INVENTION

The present invention is directed toward medical examination of patients to generate digital pre-intubation examination files for assisting successful intubations. The digital pre-intubation examination files include personal patient details including inter alia name, age, sex, contact details, etc. The digital pre-intubation examination files also include inter alia one or more of the aforesaid exterior patient anatomical measurements, facial photographic data of a patient's face, and a clinician's ratings of one or more of the standard intubation classifications. For the sake of conciseness, use of the terms a patient's face, a patient's facial structure, and the like, are intended to also refer to his neck and upper shoulders.

The present invention includes pre-intubation examination apparatus for acquiring facial photographic data of a patient's face. The facial photographic data necessarily includes at least one frontal view and at least one profile view. The facial photographic data can be in the form of one or more videos, a series of still images, or a combination of still images and one or more videos. The pre-intubation examination apparatus can be implemented either as a standalone handgun shaped pre-intubation examination probe or a pre-intubation examination accessory for use with a commercially available handheld digital device including a user interface, one or more cameras, and a display screen. Such commercially available handheld digital devices include inter alia computer tablets, smartphones, and the like. The pre-intubation examination accessory preferably includes an illumination module for illuminating a patient's face to ensure sufficient homogenous illumination for acquiring the facial photographic data and a visual reference module for projecting at least two fiduciary markers on a patient's face. The present invention also includes facial structure measurement software for automatically determining preferably the four aforesaid standard exterior patient anatomical measurements from a patient's facial photographic data.

The present invention can also include an elongated finger-shaped imaging probe for inserting into a patient's mouth for acquiring facial photographic data of his laryngeal aperture. Such facial photographic data is available for use together with a clinician's subjective rating of a patient on the Cormack-Lehane classification.

Intubation procedures are known to sometimes inadvertently damage or break one or more of a patient's teeth and particularly his upper and lower front teeth due to forces applied to a laryngoscope to assist an intubation. Such dental damage can require extensive dental repair to restore a patient's dental condition to its pre-intubation dental condition. Such dental repair can be expensive. Some dishonest patients having missing and/or broken teeth before an intubation procedure claim their missing and/or broken teeth are due to an intubation procedure. The digital pre-intubation examination files can also include a patient's dental condition information including photographic images and/or a medical report regarding missing and/or broken teeth to assess dental damage caused during an intubation procedure and counter fraudulent claims.

The pre-intubation examination of a patient in accordance with the present invention is intended to take a few minutes. The acquiring of the one or more facial photographic images is intended to take in the region of 30 seconds. The digital pre-intubation examination files are intended to be compatible with standard Electronic Medical Record (EMR) programs. The present invention is intended to facilitate organization and standardization of pre-intubation examinations and may also facilitate telemedicine consultations.

### BRIEF DESCRIPTION OF THE DRAWINGS

In order to understand the presently disclosed subject matter and to see how it may be carried out in practice, preferred embodiments will now be described, by way of non-limiting examples only, with reference to the accompanying drawings, in which:

FIG. 1 illustrates the standard Mallampati classification;

FIG. 2 illustrates the standard upper lip bite test classification;

FIG. 3 illustrates the standard Cormack-Lehane classification;

FIG. 4 illustrates measurement of a patient's thyromental distance;

FIG. 5 illustrates measurement of a patient's sternomental distance;

FIG. 6 illustrates measurement of a patient's atlanto-occipital joint extension;

FIG. 7 illustrates measurement of a patient's inter incisor gap;

FIG. 8 is a schematic view of a standalone pre-intubation examination probe for conducting pre-intubation examinations in accordance with one embodiment of the present invention;

FIG. 9 is a schematic profile view of a patient with a pair of fiduciary markers projected on his left cheek;

FIG. 10 is a schematic view of a pre-intubation examination accessory for use with a commercially available computer tablet for conducting pre-intubation examinations in accordance with another embodiment of the present invention;

FIG. 11 is a flow diagram of the pre-intubation examination method of the present invention; and

FIG. 12 is a schematic view of a patient pre-intubation examination report printed from a digital pre-intubation examination file.

### DETAILED DESCRIPTION OF THE DRAWINGS

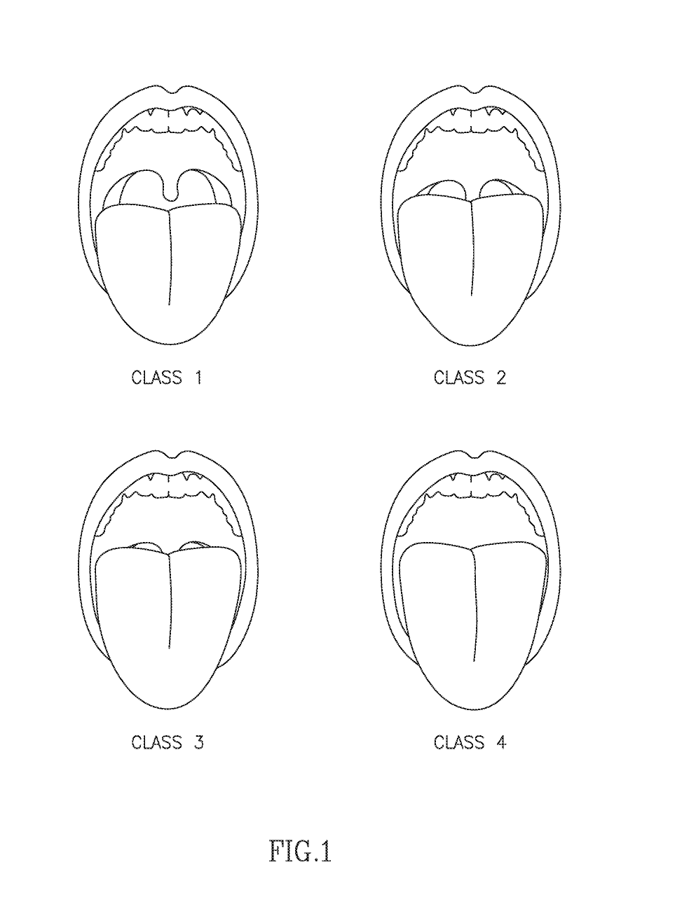

FIG. 1 shows the Mallampati classification for classifying the anatomy of a patient's oral cavity. The standard Mallampati classification includes Classes 1 to 4. Class 1 is assigned to predicted “easy to-intubate” patients. Class 4 is assigned to predicted “difficult-to-intubate” patients.

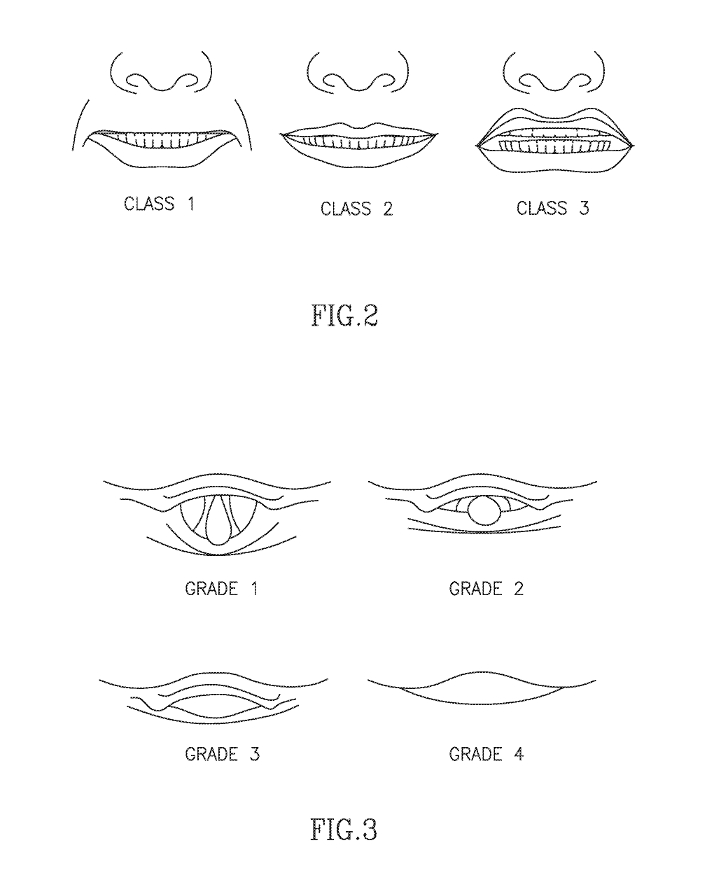

FIG. 2 shows the upper lip bite test classification for classifying the distance to which a patient can bite his upper lip with his lower incisors. The standard upper lip bite test classification includes Classes 1 to 3. Class 1 is assigned to predicted “easy-to-intubate” patients. Class 3 is assigned to predicted “difficult-to-intubate” patients.

FIG. 3 shows the Cormack-Lehane classification for classifying a patient's laryngeal aperture. The standard Cormack-Lehane classification includes Grades 1 to 4. Grade 1 is assigned to predicted “easy-to-intubate” patients. Grade 4 is assigned to predicted “difficult-to-intubate” patients.

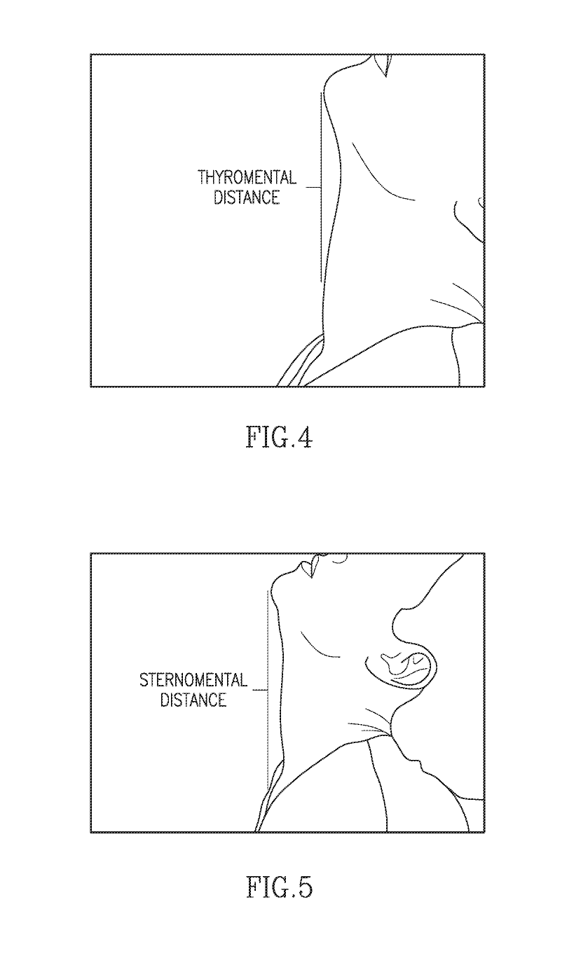

FIG. 4 shows the measurement of a patient's thyromental distance.

FIG. 5 shows the measurement of a patient's sternomental distance.

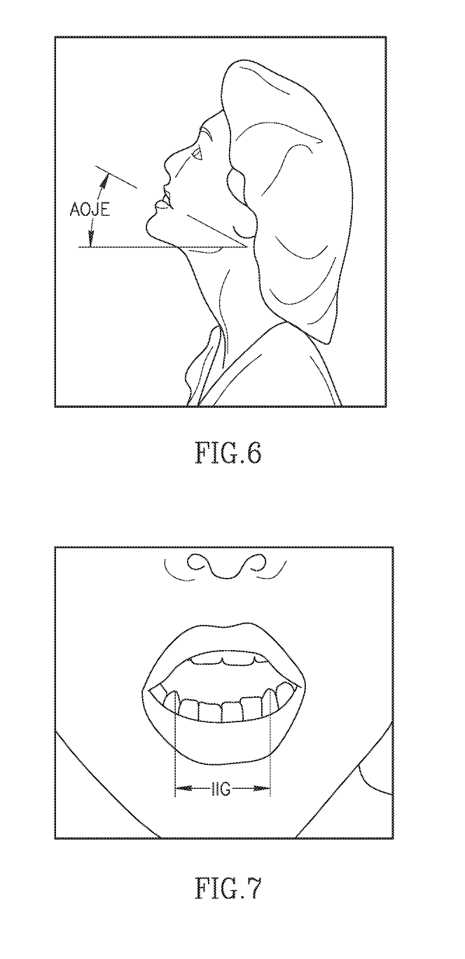

FIG. 6 show the measurement of a patient's atlanto-occipital joint extension.

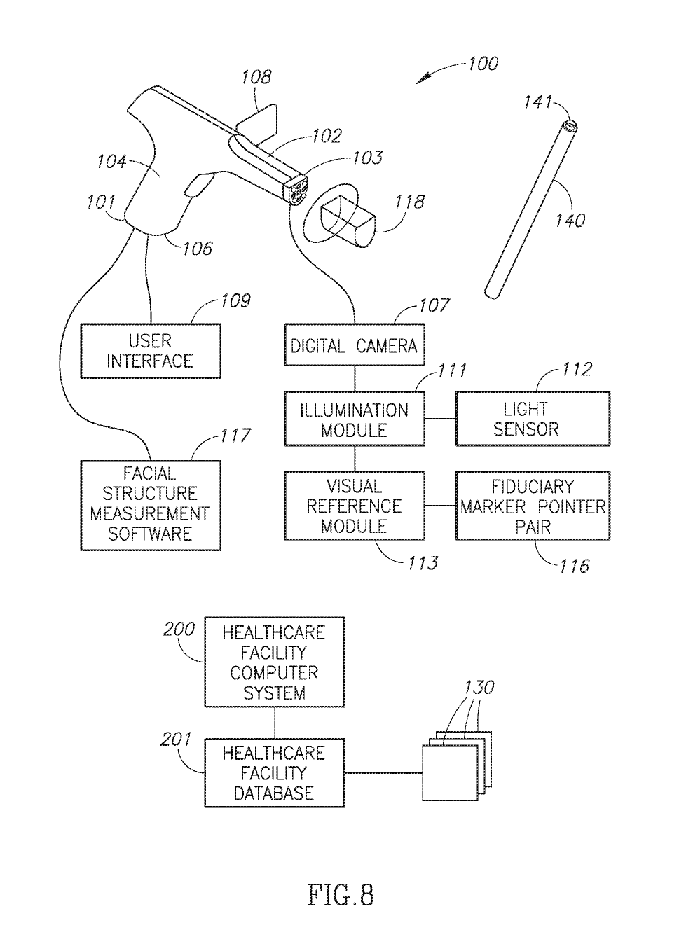

FIG. 7 show the measurement of a patient's inter-incisor gap.

FIG. 8 shows a standalone portable pre-intubation examination probe 100 for conducting pre-intubation examinations for generating digital pre-intubation examination files 130. The pre-intubation examination probe 100 is preferably handgun-shaped including a handgrip 101 and a barrel 102 with a barrel tip 103. The handgrip 101 includes a power pack compartment 104 for holding a power source 106 for operating the pre-intubation examination probe 100. The power source 106 can be in the form of replaceable batteries, a rechargeable power pack, and the like.

The barrel tip 103 includes a digital camera 107 for acquiring facial photographic data. The facial photographic data can be in the form of one or more videos and/or a series of still images. The barrel 102 supports a display screen 108 for displaying facial photographic data of a patient's face.

The pre-intubation examination probe 100 includes a user interface 109 for operating same. The user interface 109 enables a clinician to input personal patient details including inter alia name, age, sex, contact details, etc. The user interface 109 also enables a clinician to rate a patient on one or more of the three standard intubation classifications: the Mallampati classification, the upper lip bite classification and the Cormack-Lehane classification.

The barrel tip 103 includes an illumination module 111 for providing homogenous illumination for illuminating a patient's face. The illumination module 111 includes a light sensor 112 for sensing ambient light and adjusting the illumination illuminating a patient's face as a function of the ambient light.

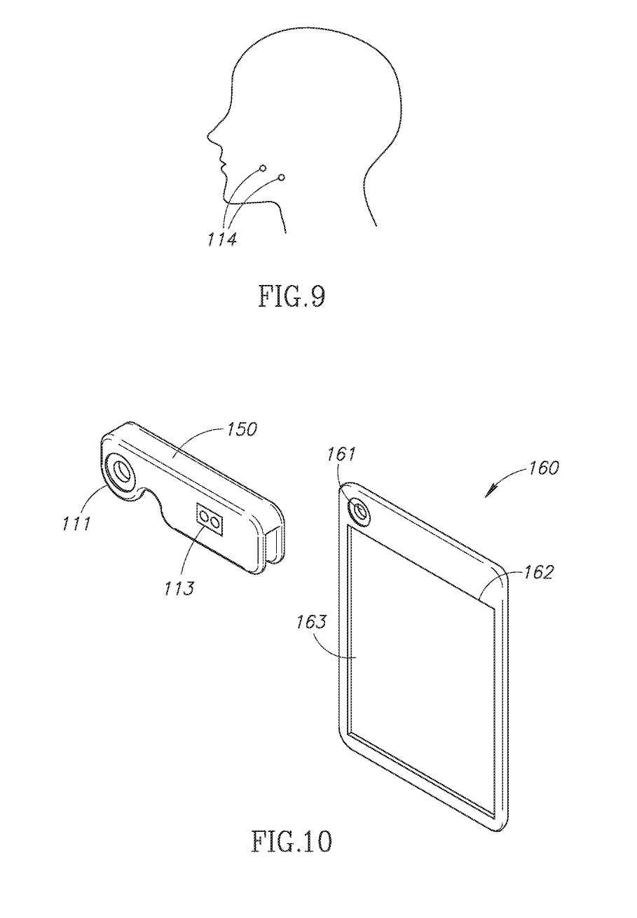

The barrel tip 103 includes a visual reference module 113 for projecting a fiduciary marker pair 114 towards a patient's face. The visual reference module 113 preferably includes a parallel fiduciary marker pointer pair 116. The parallel fiduciary marker pointer pair 116 can be constituted by VCSELs or lasers. FIG. 9 shows a patient's face with a fiduciary marker pair 114 projected on his left cheek.

The pre-intubation examination probe 100 preferably includes facial structure measurement software 117 for distinguishing physiological facial landmarks and determining objective quantitative anatomical measurements using the known distance between the fiduciary marker pair 114 as a benchmark. Such physiological facial landmarks from a patient's profile view include inter alia his nose, his chin, his throat, and the like. The physiological facial landmarks from a patient's frontal view include inter alia his mouth, teeth, tongue, uvula, soft palate, and the like. The facial structure measurement software 117 can automatically select one or more particular frames and/or still images from the facial photographic data for determining a patient's thyromental distance, sternomental distance, atlanto-occipital joint extension and inter incisor gap. Alternatively, the facial structure measurement software 117 can select particular frames and/or still images for a clinician to select the best frame or still image for the determination to be made. The facial structure measurement software 117 can determine the exterior patient anatomical measurements in real time to assist a clinician performing a pre-intubation examination procedure.

The pre-intubation examination probe 100 preferably uploads digital pre-intubation examination files 130 to a healthcare facility computer system 200. The digital pre-intubation examination files 130 can be preferably stored in a healthcare facility database 201. The pre-intubation examination probe 100 can be inserted into a docking station for uploading digital pre-intubation examination files 130 to the healthcare facility computer system 200. The pre-intubation examination probe 100 can be recharged in the docking station. Alternatively, pre-intubation examination probe 100 can be in wireless communication with the healthcare facility computer system 200. Alternatively, the healthcare facility computer system 200 can be loaded with the facial structure measurement software 117.

The pre-intubation examination probe 100 can also be in communication with a finger-shaped imaging probe 140 with a distal digital camera 141. The imaging probe 140 is intended to be inserted into a patient's mouth for acquiring facial photographic data of the patient's laryngeal aperture.

The pre-intubation examination probe 100 can be supplied with disposable protective shields 118 for placing on the barrel tip 103 for sterility purposes.

FIG. 10 shows a pre-intubation examination accessory 150 for use with a commercially available computer tablet 160 for conducting pre-intubation examinations for generating digital pre-intubation examination files 130. The commercially available computer tablet 160 includes a digital camera 161, a display screen 162 and a user interface 163. The pre-intubation examination accessory 150 includes the illumination module 111 and the visual reference module 113. The computer tablet 160 is loaded with a pre-intubation examination application for enabling operation of the computer tablet 160 in a similar manner to pre-intubation examination apparatus 100. The computer tablet 160 is also loaded with the facial structure measurement software 117. The computer tablet 160 can also be used with the finger-shaped imaging probe 140 for acquiring facial photographic data of a patient's laryngeal aperture.

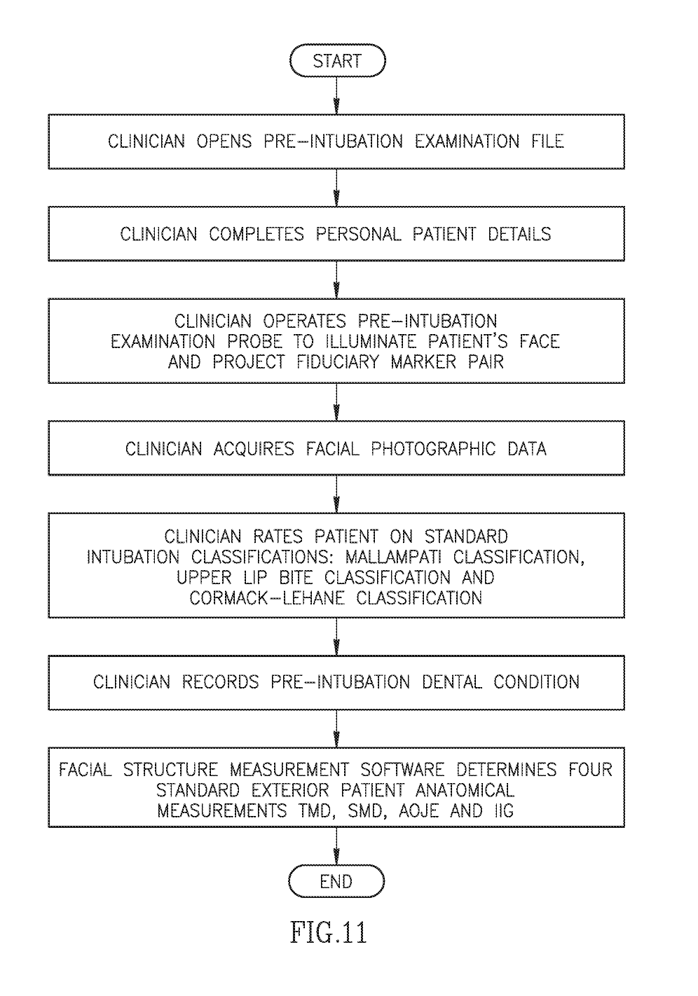

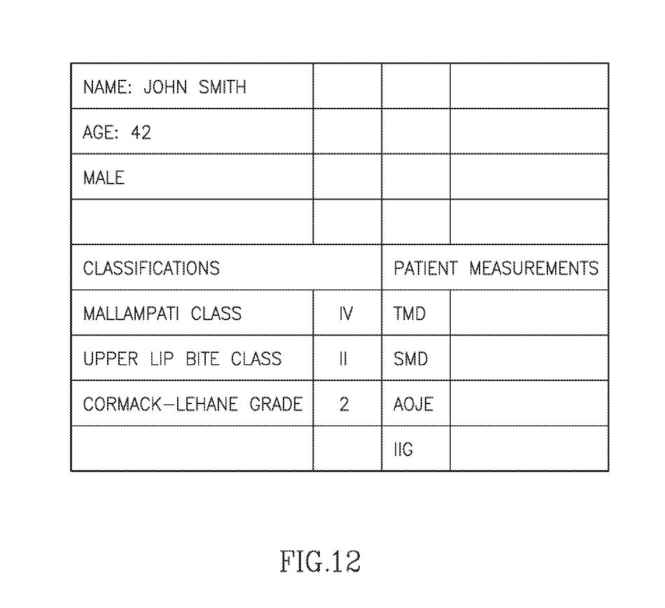

FIG. 11 shows the pre-intubation examination method for generating digital pre-intubation examination files 130 includes the following steps:

A clinician opens a new pre-intubation examination file 130 for a new patient requiring an intubation procedure. The clinician completes personal patient details for the patient including inter alia name, sex, age, contact details, etc. The clinician operates the pre-intubation examination probe 100 for illuminating the patient's face and projecting the fiduciary marker pair 114.

The clinician acquires facial photographic data of the patient's face. The clinician can acquire videos as the patients performs different head movements, for example, nodding his head up and down, turning his head to the left and the right as far as possible, etc. The clinician can acquire still images and/or a video as the patient holds his head in certain positions for determination of the standard patient measurements. The clinician can acquire still images and/or a video supporting his rating of the patient on the three standard intubation classifications. For example, the patient can be requested to open his mouth as wide as possible, to move his lower jaw to the left and right as far as possible, to protrude his tongue as far as possible and move it to the left and right as far as possible, etc. The clinician can acquire photographic data of the patient's pre-intubation dental condition including missing and/or broken teeth.

The clinician can interrupt use of the pre-intubation examination probe 100 and insert the finger-shaped imaging probe 140 into the patient's mouth for acquiring facial photographic data of the patient's laryngeal aperture.

The clinician subjectively rates the patient from being “easy to intubate” to “difficult to intubate” on at least one of the standard intubation classifications from the list of the Mallampati classification, the upper lip bite classification and the Cormack-Lehane classification and uses the user interface 109 to input his ratings.

The clinician can operate the facial structure measurement software 117 for automatically determining the four standard patient measurements: ThyroMental Distance (TMD), SternoMental Distance (SMD), Atlanto-Occipital Joint Extension (AOJE) and Inter Incisor Gap (IIG).

FIG. 12 shows a patient pre-intubation examination report 131 printed from a digital pre-intubation examination file 130. The patient pre-intubation examination report 131 includes a patient's personal details, for example, name, age, sex, contact details, etc. The patient pre-intubation examination report 131 also includes a clinician's scores of a patient on the standard intubation classifications: Mallampati classification, upper lip bite classification and Cormack-Lehane classification. The patient pre-intubation examination report 131 also includes the four standard patient measurements: ThyroMental Distance (TMD), SternoMental Distance (SMD), Atlanto-Occipital Joint Extension (AOJE) and Inter Incisor Gap (IIG). The patient pre-intubation examination report 131 also includes a clinician's findings regarding a patient's pre-intubation dental condition.

Those skilled in the art to which this invention pertains will readily appreciate that numerous changes, variations and modifications can be made without departing from the scope of the invention mutatis mutandis.

1-14. (canceled)

15. A computer-implemented method of generating a pre-intubation examination file associated with an intubation of a patient, the computer-implemented method comprising:
receiving, via at least one first digital camera, first photographic data that includes at least one frontal view and at least one profile view of the patient;
displaying, via a display screen, the first photographic data;
receiving, via a user interface operatively connected to the display screen, a selection of a rating of intubation difficulty associated with the patient for at least one of a Mallampati classification, an upper lip bite classification, and a Cormack-Lehane classification; and
populating the pre-intubation examination file with the first photographic data and the rating of intubation difficulty.

16. The computer-implemented method of claim 15, further comprising:
determining, via a computer system or a device including the user interface, a facial structure measurement of the patient including at least one of a thyromental distance, a sternomental distance, an atlanto-occiptal joint extension, and an inter incisor gap; and
populating the pre-intubation examination file with the facial structure measurement.

17. The computer-implemented method of claim 16, further comprising:
projecting, via at least one laser operatively connected to the user interface, a parallel fiduciary marker pair in a direction of a face of the patient,
wherein determining the facial structure measurement includes determining the facial structure measurement according to a location of the parallel fiduciary marker pair relative to facial landmarks on the face of the patient.

18. The computer-implemented method of claim 16, wherein determining the facial structure measurement includes determining at least the inter incisor gap.

19. The computer-implemented method of claim 15, further comprising storing, via at least one of a computer system and a device including the user interface, the pre-intubation examination file.

20. The computer-implemented method of claim 19, wherein receiving the first photographic data includes illuminating, via an illumination module including a light sensor, a face of the patient according to a level of ambient light detected by the light sensor and operating the at least one first digital camera.

21. The computer-implemented method of claim 20,
wherein the device including the user interface is a hand-held probe including the at least one first digital camera, the display screen, the illumination module including the light sensor, a hand grip, and a barrel, and
wherein receiving the first photographic data includes operating the at least one first digital camera with the hand-held probe.

22. The computer-implemented method of claim 20,
wherein the illumination module including the light sensor is provided in an accessory including at least one laser,
wherein the at least one laser is configured to project a parallel fiduciary marker pair in a direction of a face of the patient, and
wherein the accessory is configured to be removably attached to the device including the user interface.

23. The computer-implemented method of claim 15, further comprising:
receiving, via the user interface, patient details including at least one of a name, a sex, an age, and contact details, and
populating the pre-intubation examination file with the patient details.

24. The computer-implemented method of claim 15, further comprising:
receiving, via at least one second digital camera, second photographic data including a view of a laryngeal aperture of the patient;
displaying, via the display screen, the second photographic data;
receiving, via the user interface, a selection of a rating of intubation difficulty associated with the patient for the Cormack-Lehane classification; and
populating the pre-intubation examination file with the second photographic data and the rating of intubation difficulty associated with the patient for the Cormack-Lehane classification.

25. The computer-implemented method of claim 24, further comprising storing, via at least one of a computer system and a device including the user interface, the pre-intubation examination file.
